(19) United States
(12) Patent Application Publication
(10) Pub. No.: US 2020/0060540 A1
(43) Pub. Date: Feb. 27, 2020

(54) PAIRING AND EYE POLARITY DETERMINATION METHOD AND SYSTEM

(71) Applicant: Johnson & Johnson Vision Care, Inc., Jacksonville, FL (US)

(72) Inventors: Adam Toner, Jacksonville, FL (US); Scott K. Humphreys, Greensboro, NC (US); Donald K. Whitney, Melbourne, FL (US)

(21) Appl. No.: 16/112,622

(22) Filed: Aug. 24, 2018

(51) Int. Cl. A61B 3/18; A61B 3/113; A61B 3/00

(52) U.S. Cl. CPC A61B 3/18; A61B 3/0091; A61B 3/113

(57) ABSTRACT

A method including receiving sensor data during a calibration sequence for one or more a first ophthalmic device or a second ophthalmic device; determining, based on the sensor data, a change in a characteristic of one or more of the first eye or the second eye, the change relates to a calibration instruction provided to the user during the calibration sequence; determining a first polarity indicating that the first ophthalmic device is disposed adjacent one of a right eye or a left eye based on the change in the characteristic of one or more of the first eye or the second eye; determining a second polarity indicating that the second ophthalmic device is disposed adjacent the other of the right eye or left eye; and associating a first identifier with the first ophthalmic device and a second identifier with the second ophthalmic device.

### BACKGROUND

The present invention relates to ophthalmic devices having embedded controlling elements, and more specifically, to use the embedded controlling elements to conduct pairing, calibration, and customization sequences based upon user actions.

Near and far vision needs exist for all. In young non-presbyopic patients, the normal human crystalline lens has the ability to accommodate both near and far vision needs and those viewing items are in focus. As one ages, the vision is compromised due to a decreasing ability to accommodate as one ages. This is called presbyopia.

The use of Adaptive optics/powered lens products are positioned to address this and restore the ability to see items in focus. But what is required is knowing when to activate (or actuate) the optical power change. While a manual indication or use of a key fob to signal when a power change is required is one way to accomplish this change. However, leveraging anatomical/biological conditions/signals may be more responsive, more user friendly and potentially more “natural” and thus more pleasant to the wearer.

A number of things happen when we change our gaze from far to near. Our pupil size changes, our line of sight from each eye converge in the nasal direction coupled with a somewhat downward component as well. However, to sense/measure these items are difficult; one also needs to filter out certain other conditions or noise (e.g., blinking, what to do when one is lying down, or head movements).

In reference to FIG. 4, when observing an object in each eye the visual axis points toward the object or Target. Since the two eyes are spaced apart (distance b) and the focal point is in front, a triangle is formed. Forming a triangle allows the relationship of rotation angles for the left and right eyes (θL and θR, respectively) of each visual axis to the distance (Y) the object is from the eyes to be determined. Since the distance (Y) is what determines if a change in optical power is required, then the relationship between angles and the distance between the eyes and allows a system to make a decision regarding when to change the optical power. Note that the sign of the angles may be such that a counter clockwise rotation is represented by a positive angle value, and a clockwise rotation is represented by a negative angle value. Thus for normal conditions the left angle is between zero and a negative value, and the right angle is between zero and a positive value. The difference between the right and left angles may be referred to as a vergence angle.

At a minimum, sensing of multiple items may be required to remove/mitigate any false positive conditions that would indicate a power change is required when that is not the case. Use of an algorithm may be helpful. Additionally, threshold levels may vary from patient to patient, thus some form of calibration will likely be required as well.

A user may use multiple ophthalmic devices, such as one for each eye. However, an ophthalmic device may not be able to determine whether the ophthalmic device is located in the right eye or the left eye of the user. Additionally, two ophthalmic devices may not be synchronized with each other. Thus, there is a need for more sophisticated ophthalmic devices that pair multiple ophthalmic devices and determine the location of each ophthalmic device.

### SUMMARY

According to one aspect of the present invention, a method includes receiving, by one or more of a first sensor system and a second sensor system, sensor data during a calibration sequence for one or more of a first ophthalmic device or a second ophthalmic device, wherein the first sensor system is disposed on or in the first ophthalmic device, and the second sensor system is disposed in or on the second ophthalmic device; determining, based on the sensor data, a change in a characteristic of one or more of the first eye or the second eye, wherein the change relates to a calibration instruction provided to the user during the calibration sequence; determining a first polarity indicating that the first ophthalmic device is disposed adjacent one of a right eye or a left eye based on the change in the characteristic of one or more of the first eye or the second eye; determining a second polarity indicating that the second ophthalmic device is disposed adjacent the other of the right eye or left eye; and associating a first identifier with the first ophthalmic device and a second identifier with the second ophthalmic device, wherein the first identifier is indicative of the first polarity and the second identifier is indicative of the second polarity.

According to one aspect of the present invention, a system includes a first ophthalmic device configured to be disposed adjacent a first eye of a user, the first ophthalmic device comprising a first sensor system, the first sensor system comprising a first sensor and a first processor operably connected to the first sensor; a second ophthalmic device configured to be disposed adjacent a second eye of the user, the second ophthalmic device comprising a second sensor system, the second sensor system comprising a second sensor and a second processor operably connected to the second sensor; wherein the first processor and/or the second processor are/is configured to receive sensor data from one or more of the first sensor or the second sensor during a calibration sequence; the first processor and/or second processor are/is configured to determine, based on the sensor data, a change in a characteristic of one or more of the first eye or the second eye, wherein the change relates to a calibration instruction provided to the user during the calibration sequence; the first processor and/or second processor is configured to determine a first polarity indicating that the first ophthalmic device is disposed adjacent one of a right eye or a left eye based on the change in the characteristic of one or more of the first eye or the second eye; the first processor and/or second processor are/is configured to is configured to determine a second polarity indicating that the second ophthalmic device is disposed adjacent the other of the right eye or left eye, and associate a first identifier with the first ophthalmic device and a second identifier with the second ophthalmic device, the first identifier is indicative of the first polarity and the second identifier is indicative of the second polarity.

According to one aspect of the present invention, a system includes a first ophthalmic device configured to be disposed adjacent at least one of a right eye of a user or a left eye of the user; and a first sensor system disposed in or on the first ophthalmic device, the first sensor system comprising a first sensor and a first processor operably connected to the first sensor and configured to cause pairing of the first sensor system and a second sensor system disposed in or on a second ophthalmic device, the first processor is configured to determine whether the first ophthalmic device is disposed adjacent the left eye of the user or the right eye of the user based on a calibration sequence provided to the user.

### BRIEF DESCRIPTION OF THE OF THE DRAWINGS

FIG. 1 shows an exemplary implementation according to an embodiment of the present invention.

FIG. 2 shows a flowchart according to an embodiment of the present invention.

FIG. 3 shows another exemplary implementation according to an embodiment of the present invention.

FIG. 4 shows an example of focus determination.

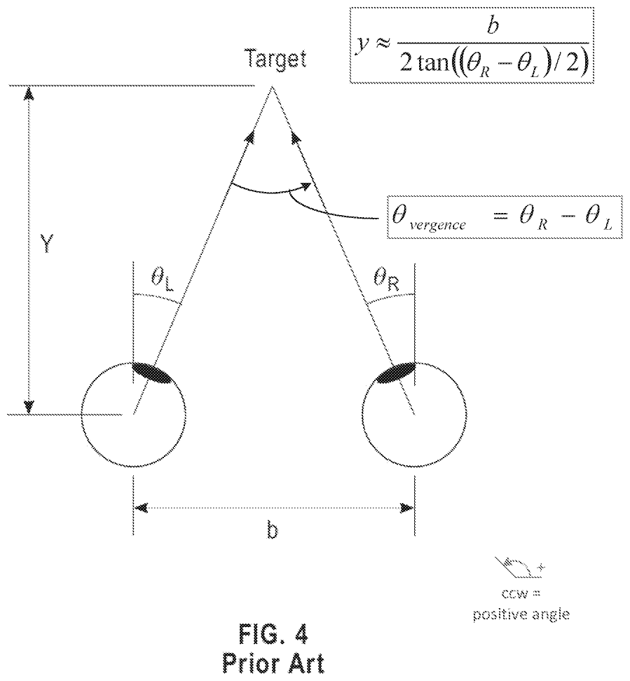

FIG. 5 shows another flowchart according to an embodiment of the present invention.

FIG. 6 shows another flowchart according to an embodiment of the present invention.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting. As will be appreciated by one skilled in the art, aspects of the present invention may be embodied as a system, method or computer program product.

The present methods and systems relate to an ophthalmic system comprising one or more ophthalmic devices, such as a system comprising at least one ophthalmic device for each eye of a user. In such a system, pairing and calibration of multiple ophthalmic devices can be important.

Pairing and calibration can comprise at least three areas: 1) Finding or discovery of other ophthalmic devices as well as other devices (e.g., a phone, a tablet), 2) using a security protocol that will reduce the chances of unwanted sharing of information or control, and 3) calibration activities such as clock synchronization, eye determination, dominant control (e.g., primary-secondary, master-slave), and/or other features. Pairing can comprise, overlap with, and/or be part of a calibration of the ophthalmic devices.

Calibration can be used (e.g., after or during paring) to configure ophthalmic devices to be more accurate. Because everyone's eyes are a bit different, (e.g., pupil spacing and location, lens-on-eye position, etc.), even at a fixed close distance, initial vergence angles will differ from patient to patient. It is important once ophthalmic devices (e.g., lenses) are placed in or on the eye to calibrate what the initial vergence angle is, so that differences in this angle can be assessed while in service. This value can be used for subsequent calibration calculations. Accurate calculation of vergence angles (e.g., or other vergence parameters) may be dependent on a determining which eye an ophthalmic device is located on or in. Association of an ophthalmic device with a particular eye (e.g., left, right) or side of the body can be referred to as polarity. For example, a polarity parameter can have two values (e.g., 0 and 1, left and right, true and false), one indicating that the ophthalmic device is disposed in or on the right eye of a user and another indicating that the ophthalmic device is disposed in or on the left eye of the user.

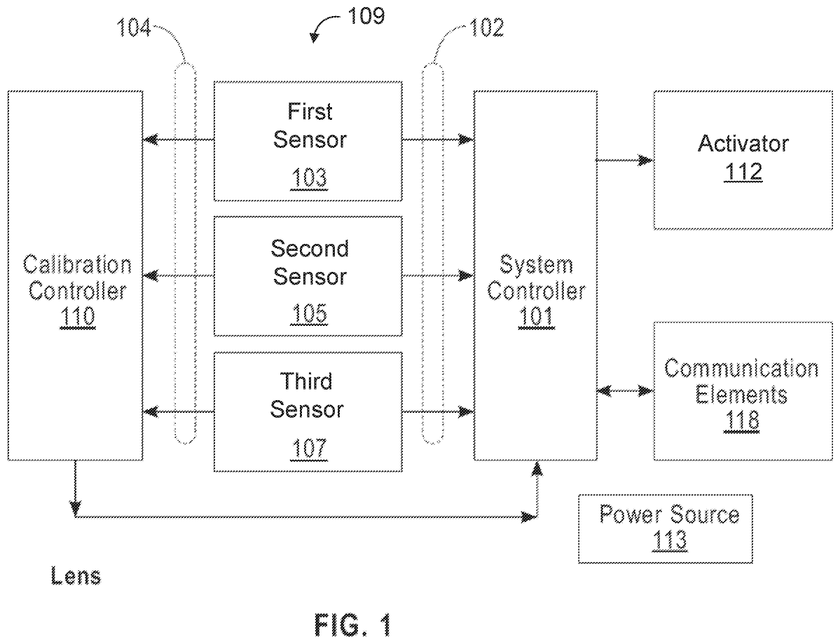

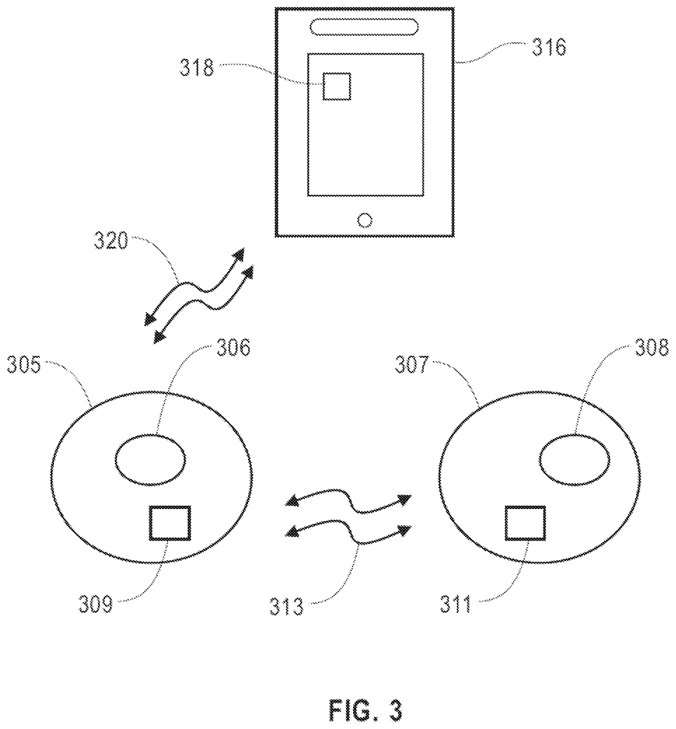

Now referring to FIG. 1, an exemplary implementation shows a system (e.g., or sensor system) according to an embodiment of the present invention. The system can be disposed in or on an ophthalmic device. The ophthalmic device can comprise a contact lens or an implantable lens, or a combination of both. The ophthalmic device, such as a contact lens, can be configured to be disposed adjacent an eye of a user. The contact lens comprises a soft or hybrid contact lens. The ophthalmic device can be part of system of at least two ophthalmic devices, as shown in FIG. 3.

A system controller 101 controls an activator 112 (e.g., lens activator) that changes the adaptive optics/powered lens (see FIG. 3) to control the ability to see both near and far items in focus. The system controller 101 can comprise a processor, memory, and/or the like. The system controller 101 (e.g., the processor) can be operably coupled to a sensor element 109. The system controller 101 receives signals 102 (e.g., data signals, control signals) from the sensor element 109.

The sensor element 109 can comprise a plurality sensors (103, 105 and 107). Examples of sensors can comprise a multidimensional sensor, a capacitive sensor, an impedance sensor, an accelerometer, a temperature sensor, a displacement sensor, a neuromuscular sensor, an electromyography sensor, a magnetomyography sensor, a phonomyography, or a combination thereof. The plurality of sensors (103, 105 and 107) can comprise a lid position sensor, a blink detection sensor, a gaze sensor, a divergence level sensor, an accommodation level sensor, a light sensor, a body chemistry sensor, neuromuscular sensor, or a combination thereof. The plurality of sensors (103, 105 and 107) can comprise one or more contacts configured to make direct contact with tear film of an eye of the user.

As an illustration, the plurality of sensors (103, 105 and 107) can comprise a first sensor 103, such as a first multidimensional sensor that includes an X-axis accelerometer. The plurality of sensors (103, 105 and 107) can comprise a second sensor 105, such as a second multidimensional sensor that includes a Y-axis accelerometer. The plurality of sensors (103, 105 and 107) can comprise a third sensor 107, such as a third multidimensional sensor that includes a Z-axis accelerometer. As another embodiment, the three axis accelerometers can be replaced by a three-axis magnetometer. However, other configurations may be used including a 3-axis accelerometer, a magnetometer, and a light or temperature sensor. Calibration would be similar because each axis would potentially require calibration at each extreme of each axis. The plurality of sensors (103, 105 and 107) further provide calibration signals 104 to a calibration controller 110. The calibration controller 110 conducts a calibration sequence based on the calibration signals from the plurality of sensors (103, 105 and 107) as a result of user actions which is sensed by the plurality of sensors (103, 105 and 107) and provides calibration control signals 102 to the system controller 101. The system controller 101 further receives from and supplies signals to communication elements 118. Communication elements 118 allow for communications between user lens and other devices such a near-by smartphone. A power source 113 supplies power to all of the above system elements. The power source can comprise a battery. The power sources may be either a fixed power supply, wireless charging system, or may be comprised of rechargeable power supply elements. Further functionality of the above embedded elements is described herein.

The system controller 101 can be configured to perform a pairing procedure. For example, the system can comprise at least two ophthalmic devices, as shown later in FIG. 3. For purposes of illustration multiple ophthalmic devices are described, one or more (or each) of which can be an ophthalmic device as shown in FIG. 1. For example, a first ophthalmic device can be configured to be disposed adjacent a first eye of a user. As illustrated in FIG. 1, the first ophthalmic device can comprise a first sensor system. The first sensor system can comprise a first sensor and a first processor operably connected to the first sensor. A second ophthalmic device can be configured to be disposed adjacent a second eye of the user. The second ophthalmic device can comprise a second sensor system. The second sensor system can comprise a second sensor and a second processor operably connected to the second sensor.

The pairing procedure can comprise one or more phases, such as a discovery phase, a security phase, and a synchronization phase. The discovery phase can comprise listening (e.g., at least periodically, or upon a triggering event, such as a particular movement or receiving a message). For example, one or more of the ophthalmic devices can broadcast (e.g., wirelessly or ultrasonically) a beacon indicating the presence of the ophthalmic device. The beacon can comprise any kind of data, such as a device identifier (e.g., of the ophthalmic device), associated user identifier, device capabilities, and/or the like. If one ophthalmic device detects the beacon of another ophthalmic device, then the two ophthalmic devices can determine to enter the synchronization phase. For example, if the device identifier, associated user identifier, device capabilities, and/or other information matches criteria (e.g., information stored on the receiving ophthalmic device), then the two ophthalmic devices can continue the pairing procedure by entering the security phase. For example, each ophthalmic device may transmit a beacon at a random time and listen for a beacon or an acknowledge signal from another ophthalmic device. When a beacon is detected at one ophthalmic device it may transmit an acknowledgement signal at a predetermined time delay after reception of the beacon. The ophthalmic devices may be configured to listen for beacons at any time when not transmitting or even during transmission, and/or to listen for acknowledge signals in a time frame around a predetermined delay from transmitting a beacon.

With gesture recognition, a calibration phase may be entered on detection of a certain sequence of movements, e.g. looking up and to the left for more than 3 seconds. Additionally or alternatively, devices may be first powered up by sensing light and then may enter the pairing phase. Any suitable modulation method could be used, such as on-off-keying or FM modulation. An ALOHA protocol is typically used for initial asynchronous communications

During the security phase, the two ophthalmic devices can establish a secure connection based on a security protocol. For example, the security protocol can comprise encryption, the use of digital certificates, and/or the like. For example, the security protocol can be based on symmetric-key cryptography, public-key cryptography, end-to-end encryption, and/or the like.

During the synchronization phase, the ophthalmic devices can be configured to synchronize clocks. The ophthalmic devices can be configured to synchronize internal oscillators (e.g., local oscillator circuit) with each other. This allows internal timers to be synchronized, which allows each ophthalmic device to periodically enter low power mode until later powering up and communicating or perform other tasks. For example, the ophthalmic devices can communicate and/or process information based on a predefined schedule. As another example, ophthalmic devices can communicate and/or process information based on a dynamically negotiated time interval (e.g., or schedule). The dynamically negotiated time interval may vary based on recent activity occurring in or detected by sensors in one or more of the ophthalmic devices.

During (e.g., or following) the synchronization phase, one or more of the ophthalmic devices can be configured to perform any calibration procedure as described herein. The calibration can comprise determining which of the ophthalmic devices will be primary, and which of the ophthalmic devices will be secondary (e.g., the secondary may receive instructions from the primary). The calibration can comprise determining polarity indicating in which of the user's eye each of the ophthalmic devices is located.

In the context of using sensors to determine vergence, for example using accelerometers, there are opportunities to calibrate. Offsets, due to the micro-electromechanical systems (MEMS) and/or due to the electronics, mounting on the interposer, etc. can cause variations with the algorithms and thus cause some errors in the measurement of vergence. In addition, human anatomy from person to person, is different. For instance, eye to eye space can vary from 50 to 70 mm and can cause a change in trigger points based on eye spacing alone. So there is a need to take some of these variables out of the measurement, thus calibration and customization performed by the current embodiment when the lens are on the user. This serves to improve the user experience by both adding the preferences of the user and to reduce the dependencies of the above-mentioned variations.

The plurality of sensors (103, 105 and 107) can measure acceleration both from quick movements and from gravity (9.81 m/s²). The plurality of sensors (103, 105 and 107) may produce a code that is in units of gravity (g). The determination of vergence depends on the measurement of gravity to determine position, but other methods may depend on the acceleration of the eye. There are going to be differences and inaccuracies that will require base calibration before use calibration.

The current embodiment uses three sensors on each ophthalmic device. However, calibration may be done using two sensors, e.g., the first sensor 103 (e.g., X-axis accelerometer) and the second sensor 105 (e.g., Y-axis accelerometer). Other combinations of the same or different sensors may be used. In various embodiments, each accelerometer has a full scale plus, full scale minus, and zero position. The errors could be offset, linearity, and slope (or gain) errors. A full calibration would correct all three error sources for all axes sensors being used.

One way to calibrate the sensors is to move them such that each axis is completely perpendicular with gravity, thus nominally reading 1 g. Then the sensor would be turned 180 degrees and it should read −1 g. From two points, the slope and intercept can be calculated and used to calibrate. This is repeated for the other two sensors. This is an exhaustive way of calibrating the sensors and thus calibrating the vergence detection system. A calibration offset value cal_offset may be calculated by comparing a measured value meas_value to an expected value or “reference value” ref_value for a given calibration orientation. In some embodiments the measured value meas_value may comprise an average of a number of measurements, e.g. 3 or more, to account for user movement and noise/vibration. The calibration offset value cal_offset may be calculated as the difference of the measured value meas_value minus the reference value ref_value. In an operating mode, new measurements may be calibrated by subtracting the calibration offset value cal_offset from the measured value meas_value to create a calibrated value cald_value. In a similar manner separate calibration offsets could be developed for each axis.

Cal offset=meas_value−reference_value.

Cald value=meas_value−cal_offset.

At the reference orientation, it should be appreciated that:

Cal'd value=meas_value−(meas_value-ref_value)=ref_value.

When using this manner of calibration, note that gain or slope calibration utilizes two measurements at different angles for a given axis, e.g. looking at 45 deg and 90 deg and expecting 0.707 g and 1.0 g.

Another way is to reduce the calibration effort for the ophthalmic device is to have the wearer perform just one or two steps. One way is to have the wearer look forward, parallel to the floor, at a distant wall. Measurements taken at this time can be used to determine the offset of the vertical axis. Then have the wearer look at a point on the floor or ground half-way between the wearer and the wall without moving their head. This orientation rotates the eyes and lenses to 45 degrees from horizontal. Measurements taken at this time can be used to determine the offset of the horizontal axis by comparing the horizontal axis measurement to the expected reference value of 0.707 g. Determining the offset for each axis in the area where the user will spend most of the time provides a greater benefit to maintain accuracy.

As a further explanation, the user can be instructed to look at one or more reference locations. The one or more reference locations can comprise a first reference location and a second reference location further away from the user than the first reference location. For example, the user can receive one or more calibration instructions (e.g., as part of a calibration sequence). The calibration instruction can be provided to the user via a mobile device. The calibration sequence can be for calibrating polarity of multiple ophthalmic devices.

A first ophthalmic device and/or a second ophthalmic device can be configured to perform a polarity calibration. As explained further herein, a first ophthalmic device can be configured to be disposed adjacent a first eye of a user. The first ophthalmic device can comprise a first sensor system. The first sensor system can comprise a first sensor and a first processor operably connected to the first sensor. Similarly, the second ophthalmic device can be configured to be disposed adjacent a second eye of the user. It should be noted that use of the phrases “configured to be disposed adjacent a second eye” and “configured to be disposed adjacent a first eye” do not imply that the second ophthalmic device and/or first ophthalmic device are only configured for one specific eye. Instead, it should be understood that the first eye and second eye can be any appropriate eye, and the terms first and second are only used for purposes of explanation. The second ophthalmic device can comprise a second sensor system. The second sensor system can comprise a second sensor and a second processor operably connected to the second sensor. Before calibration, neither the first sensor system nor the second sensor system may be configured for a specific eye of the user. Accordingly, the first sensor system and/or the second sensor system can be calibrated with the correct eye polarity (e.g., left or right) for more accurate calculations.

The first processor and/or the second processor can be configured to receive sensor data from one or more of the first sensor or the second sensor during a calibration sequence. The first processor and/or the second processor can be configured to determine, based on the sensor data, a change in a characteristic of one or more of the first eye or the second eye. The change can relate to a calibration instruction provided to the user during the calibration sequence. The change can represent a change from the user looking at the first reference point to looking at the second reference point. For example, the first processor and/or the second processor can be configured to determine a direction that the user moves one or more of the first eye or the second eye (e.g., due to moving from the looking at the first reference location to looking at the second reference location). The first processor and/or the second processor can be configured to determine a change in a vergence angle of the first eye and/or the second eye (e.g., due to moving from the looking at the first reference location to looking at the second reference location). [Example of a polarity calibration: The user may be instructed to look at a first reference point at a far distance where the eyes are looking nearly parallel to each other, e.g. at a wall more than 3 meters or 10 feet away. The first sensor system and the second sensor system may record first sensor measurements in this first position. Then the user may be instructed to look at a second reference point at a close distance similar to that used for reading, e.g. at the palm of their hand or at an object they hold in their hand. The first sensor system and the second sensor system may record second sensor measurements in this second position. Then the first sensor system may calculate a first and second gaze angle for the first sensor in each position, and the second sensor system may calculate a first and second gaze angle for the second sensor in each position. The gaze angles for both positions of both sensors are then transmitted to a third device, or the gaze angles for both positions of one sensor may be transmitted from one of the first or second sensor systems (or controllers) to the other of the first or second sensor systems (or controllers). The gaze angles when looking at the second reference point (or at a close distance) will be such that the wearers left eye is rotated to the right, and the wearer's right eye is rotated to the left. By comparing the expected polarity or sign of the angles, based on the design of the lens and the convention chosen for positive angles of rotation, the polarity of the lenses may be determined. For example, if a positive angle is expected for the left eye and a negative angle for the right eye, then a sensor system can determine to which eye it is adjacent simply by checking the sign of the measured angle.

The first processor and/or the second processor can be configured to determine a first polarity indicating that the first ophthalmic device is disposed adjacent one of a right eye or a left eye based on the change in the characteristic of one or more of the first eye or the second eye. The first processor and/or the second processor can be configured to determine a second polarity indicating that the second ophthalmic device is disposed adjacent the other of the right eye or left eye. The first processor and/or the second processor can be configured to associate a first identifier with the first ophthalmic device and associate a second identifier with the second ophthalmic device. The first identifier can be indicative of the first polarity, and the second identifier can be indicative of the second polarity.

After completing calibration, the first ophthalmic device and/or the second ophthalmic device can resume normal operation. For example, the first processor and/or the second processor can be configured to receive additional sensor data and process the additional sensor data. The additional sensor data can be processed based on one or more of the first polarity and the second polarity.

Further customization can be performed during and/or after calibration. Given that everyone is a little different, customizable features can prove a better user experience for all users than a one size fits all approach. When using the ophthalmic devices with just two modes, accommodation and gaze, then the point where this is a switch from gaze to accommodation one can have several parameters in addition to the switching threshold that would affect the user experience.

The threshold going from gaze to accommodation is depended on the user, the user's eye condition, the magnification of the ophthalmic device, and the tasks. For reading, the distance between the eye and book is about 30 cm, where computer usage is about 50 cm. A threshold set for 30 cm might not work well for computer work, but 50 cm would work for both. However, this longer threshold could be problematic for other tasks by activating too early, depending on the magnification and the user's own eye condition. Thus, the ability to alter this threshold, both when the ophthalmic devices is first inserted and at any time afterwards as different circumstances could require different threshold points, provides the user customization to improve visibility, comfort and possibly safety. Even having several present thresholds is possible and practical, where the user would choose using the interfaces described here to select a different threshold. In addition, the user could alter the threshold or other parameters by re-calibrating per the embodiments of the present invention as described hereafter.

Still referring to FIG. 1, switching from gaze to accommodation, the system uses the threshold as the activation point. However, going from accommodation to gaze the threshold is shifted to a greater distance, which is called hysteresis. Accounting for hysteresis is added in order to prevent uncertainty when the user is just at the threshold and there are small head movements which may cause it to switch from gaze to accommodation to gaze, etc. Most likely, the user will be looking at a distant target when he wants to switch, so the changing of the threshold is acceptable. The hysteresis value can be determined in several ways: one, the doctor fitting the ophthalmic devices can change it, two, the user can change this value via an ophthalmic device interface, and three, an adaptive algorithm can adjust it based on the habits of the user.

Custom Modes are common now in cars, i.e. sport, economy, etc. which allow the user to pick a mode based on anticipated activity where the system alters key parameters to provide the best experience. Custom Modes are also integrated into the ophthalmic devices of the current embodiments. Calibration and customization settings can be optimized for a given mode of operation. If the user is working in the office, it is likely that the user will need to go between states (e.g., gaze and accommodation), or even between two different vergence distances because of the nature of the tasks. Changes in the threshold, hysteresis, noise immunity, and possible head positions would occur to provide quicker transitions, possible intermediate vergence positions, and optimization for computer tasks, as well as, tasks that there is a lot if switching between gaze and accommodation. Thus, options to switch the ophthalmic device into different modes to optimize the ophthalmic device operation can provide an enhanced user experience. Furthermore, in an “Exercise” mode, the noise filtering is increased to prevent false triggering and additional duration of positive signal is required before switching to prevent false switching of the ophthalmic devices being triggered by stray glances while running. A “Driving” mode might have the ophthalmic device being configured for distant use or on a manual override only. Of course, various other modes that could be derived as part of the embodiments of the present invention.

In today's world, the smart phone is becoming a person's personal communications, library, payment device, and connection to the world. Apps for the smartphone cover many areas and are widely used. One possible way to interact with the ophthalmic device of the present invention is to use a phone app. The app could provide ease of use where written language instructions are used and the user can interact with the app providing clear instructions, information, and feedback. Voice activation options may also be included. The app can provide one or more calibration instructions to the user as part of a calibration sequence. For instance, the app provides the prompting for the sensor calibrations by instructing the user to look forward and prompting the user to acknowledge the process start. The calibration sequence can comprise a first calibration instruction to look at a first reference point. The calibration sequence can comprise a second calibration instruction to look at a second reference point. The app could provide feedback to the user to improve the calibration and instruct the user what to do if the calibration is not accurate enough for optimal operation. This would enhance the user experience.

Additional indicators, if the smart phone was not available, can be simple responses from the system to indicate start of a calibration cycle, successful completion, and unsuccessful completion. Methods to indicate operation include, but are not limited to, blinking lights, vibrating haptics drivers, and activating the ophthalmic device. Various patterns of activation of these methods could be interpreted by the user to understand the status of the ophthalmic device. The user can use various methods to signal the ophthalmic device that he/she is ready to start or other acknowledgements. For instance, the ophthalmic device could be opened and inserted into the eyes awaiting a command. Blinks or even closing one's eyes could start the process. The ophthalmic device (e.g., lens) then would signal the user that it is starting and then when it finishes. If the ophthalmic device requires a follow-up, it signals the user and the user signals back with a blink or eye closing.

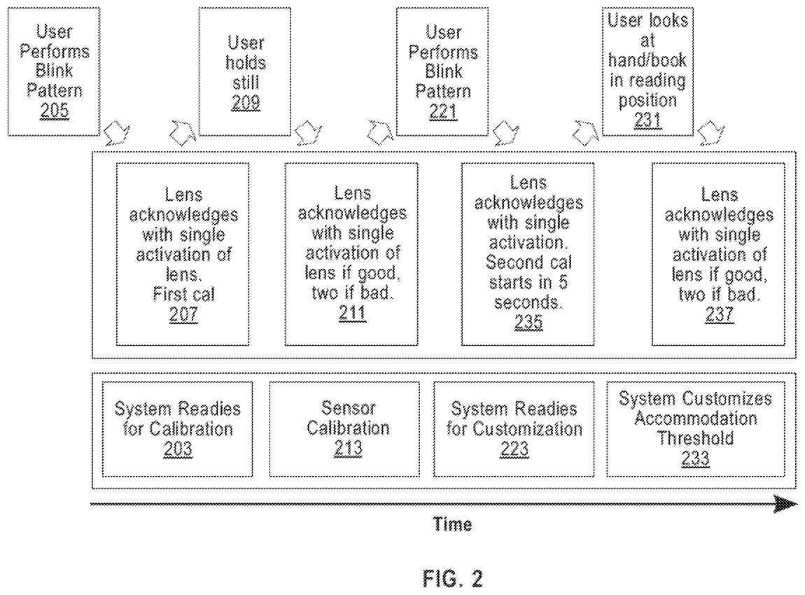

Referring to FIG. 2, one method according to an embodiment of the present invention is depicted. The process starts at an initial time (far left of the figure) and proceeds forward in time. Once the ophthalmic devices (see FIG. 3) are inserted, the system readies for calibration 203. The user performs a blink pattern 205. The ophthalmic device (e.g., lens) acknowledges with a single activation of the ophthalmic device 207 as part of a first calibration. The user holds still 209 as the system and the sensor calibration 213 starts. The ophthalmic device acknowledges with a single activation of the ophthalmic device if the first stage of calibration is good 211. If the initial calibration is bad, then the ophthalmic device acknowledges with a double activation 211. If the calibration is bad, then the user must restart the calibration process 205. After the initial calibration, the system is ready for customization 223. The user conducts another blink pattern 221. The ophthalmic device may acknowledge with a single activation of the ophthalmic device and a second calibration, customization, is started in some fixed time 235 as part the system customization accommodation threshold 233. However, the activation may be pulsed such as alternating on/off for fixed periods of time or toggling states for a time period and then returning to an original state. The user then looks at either their hand or a book at reading position 231. The ophthalmic device acknowledges with a single activation of the ophthalmic device if the second stage of calibration customization is good 237. If the second stage of calibration customization is bad, then the user must restart the calibration customization process 221. Once the ophthalmic device acknowledges with a single activation of the ophthalmic device that the second stage of calibration customization is good 237 the system has the completed customization accommodation calibration and the ophthalmic devices are ready for full use by the user. It should be noted that such method is not limited to the accommodation calibration. A similar approach can be used for other calibration operations. For example, step 233 can be adapted to include an eye polarity determination.

Other embodiments to customize the threshold can be accomplished. One way is to have the user's doctor determine the comfortable distance for the user by measuring the distance between the eyes of the patent, the typical distance for certain tasks, and then calculate the threshold. From there, using trial and error methods, determine the comfortable distance. Various thresholds can be programmed into the ophthalmic device and the user can select the task appropriate threshold.

Another method is to allow the user to select to perform pairing and/or calibration himself.

The calibration can comprise determining the polarity value as well as other values, such as a user customized accommodation threshold. The ophthalmic device can use the same system that it uses to measure the user's relative eye position to set the accommodation threshold. Where the user's preference of when to activate the extra ophthalmic device power. There is an overlap where the user's eyes can accommodate unassisted to see adequately and where the user's eyes also can see adequately with the extra power when the ophthalmic device is active. At what point to activate determined by user preference. Providing a means for the user to set this threshold, improves the comfort and utility of the ophthalmic devices. A procedure follows this sequence:
The user (e.g., wearer) prompts the system to start the sequence. Initially the system could prompt the user as a part of the initial calibration and customization;
The ophthalmic devices are activated. The ability to achieve a comfortable reading position and distance requires the user to actually see the target, thus the ophthalmic devices are in the accommodation state;
The user focuses on a target which is at a representative distance while the system determines the distance based on the angles of the eyes by using the sensor information (accelerometers or magnetometers); after several measurements and noise reduction techniques the system calculates a threshold and indicates that it has finished,
The new threshold has been determined. A slight offset is subtracted to effectively place the threshold a little farther away, thus creating hysteresis. This is necessary to move the threshold slightly longer (angle slightly lower) in order to guarantee when the user is in the same position, the system will accommodate even with small head or body position differences.

The value of this hysteresis could be altered by an algorithm that adapts to user habits. For example, if the wearer often moves an object close to trigger lens activation and then backs the object further away for a period of time, e.g. to read or view the object, then the [system] may adapt the threshold to be slightly further away so the wearer does not need to bring the object quite so close, thus providing a more natural experience. Also, the user could manually change the value if the desired by having the system prompt the user to move the focus target to a position that the user does not want the ophthalmic device to activate all the while focusing on the target. The system would deactivate the ophthalmic device and then determine this distance. The Hysteresis value is the difference in the deactivate distance and the activate distance. Ophthalmic devices are now on dependent on the new threshold and hysteresis values.

Additionally, the system can determine a polarity value based on a change in a characteristic of a user. For example, the change can be determined by comparing sensor information from before and after the user focuses on the target. For example, an initial and final vergence value (e.g., angle) can be determined. A direction of movement from an accelerometer can be determined. Then, the polarity can be assigned based on comparing the change to expected values (e.g., thresholds) associated with corresponding polarities.

To have a good user experience, the user can receive confirmation that the system has completed any adjustments or customization. In addition, the system can be configured to determine if the user performed these tasks properly and if not, and then request that the user preforms the procedure again. Cases that prevent proper customization and adjustment may include excessive movement during measurement, head not straight, lens out of tolerance, etc. The interactive experience will have far less frustrated or unhappy users.

Feedback can be given through various means. Using a phone app provides the most flexibility with the screen, cpu, memory, internet connection, etc. The methods as discussed for calibration per the embodiments of the present invention can be done in conjunction with the use of a smartphone app with use of the communication elements as described in reference to FIG. 1 and with reference to FIG. 3 hereafter.

As a part of continual improvement for the ophthalmic devices, data for the ophthalmic devices can be collected and sent back to the manufacturer (anonymously) via the smartphone app to be used to improve the product. Collected data includes, but not limited to, accommodation cycles, errors, frequency that poor conditions occur, number of hours worn, user set threshold, etc.

Other methods to indicate operation include, but not limited to, blinking lights, vibrating haptics drivers, and activating the ophthalmic devices. Various patterns of activation of these methods could be interpreted by the user to understand the status of the ophthalmic device.

Referring now to FIG. 3, shown is another exemplary implementation according to an embodiment of the present invention in which sensing and communication may be used to communicate between a pair of ophthalmic devices (305, 307), such as contact lenses. Pupils (306, 308) are illustrated for viewing objects. The ophthalmic devices (305, 307) include embedded elements, such as those shown in FIG. 1. The embedded elements (309, 311) included for example 3-axis accelerometers/magnetometers, lens activators, calibration controller, a system controller, memory, power supply, and communication elements as is described in detail subsequently. A communication channel 313 between the two ophthalmic devices (305, 307) allows the embedded elements to conduct calibration between the ophthalmic devices (305, 307). Communication may also take place with an external device, for example, spectacle glasses, key fob, dedicated interface device, or a smartphone.

As an example, communication between the ophthalmic devices (305, 307) can be important to detect or determine proper calibration. Communication between the two ophthalmic devices (305, 307) may take the form of absolute or relative position, or may simply be a calibration of one ophthalmic device to another if there is suspected eye movement. If a given ophthalmic device detects calibration different from the other ophthalmic device, it may activate a change in stage, for example, switching a variable-focus or variable power optic equipped contact lens to the near distance state to support reading. Other information useful for determining the desire to accommodate (focus near), for example, lid position and ciliary muscle activity, may also be transmitted over the communication channel 313. It should also be appreciated that communication over the channel 313 could comprise other signals sensed, detected, or determined by the embedded elements (309, 311) used for a variety of purposes, including vision correction or vision enhancement.

The communications channel (313) comprises, but is not limited to, a set of radio transceivers, optical transceivers, or ultrasonic transceivers that provide the exchange of information between both ophthalmic devices and between the ophthalmic devices and a device such as a smart phone, FOB, or other device used to send and receive information. The types of information include, but are not limited to, current sensor readings showing position, the results of system controller computation, synchronization of threshold and activation. In addition, the device or smart phone could upload settings, sent sequencing signals for the various calibrations, and receive status and error information from the ophthalmic devices.

The communications channel (313) can be established via a pairing procedure. The communications channel (313) can be a secure communication channel. For example, the communications channel (313) can be encrypted and/or may comprise a secure connection based on a security protocol. For example, the communications channel (313) can be established based on security protocol. The security protocol can comprise encryption, the use of digital certificates, and/or the like. For example, the security protocol can be based on symmetric-key cryptography, public-key cryptography, end-to-end encryption, and/or the like.

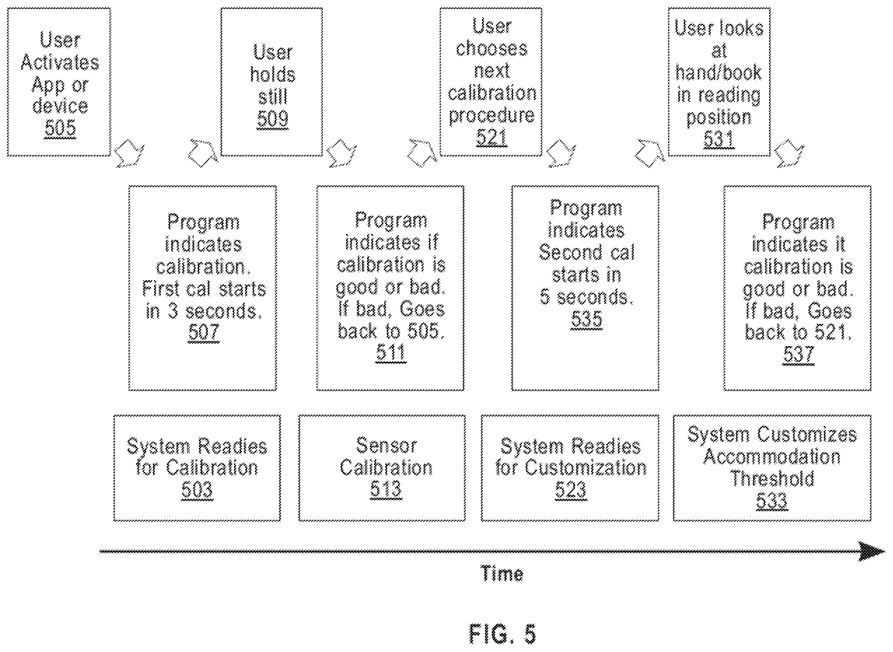

Still referring to FIG. 3, the ophthalmic devices (305, 307) further communicate with a smart phone (316) or other external communication device. Specifically, an app 318 on the smart phone (316) communicates to the ophthalmic devices (305, 307) via a communication channel (320). The functionally of the app (318) follows the process as outlined with referenced to FIG. 5 (described hereafter) and instructs the user when to perform the required eye movements. In addition, the device or smart phone (316) could upload settings, sent sequencing signals for the various calibrations, and receive status and error information from the ophthalmic devices (305, 307).

Referring to FIG. 5, another method according to an embodiment of the present invention is depicted. The process starts at an initial time (far left of the figure) and proceeds forward in time. Once the ophthalmic devices (see FIG. 3) are inserted, the system readies for calibration 503. User activates App or device 205. The app program indicates calibration and the first calibration starts in 3 seconds 507 as part of a first calibration. The user holds still 509 as the system and the sensor calibration 513 starts. The program indicates if calibration is good or bad 511. If calibration is bad the program restarts and goes back (to step 505) 511. After the initial calibration, the system is ready for customization 523. The user chooses the next calibration procedure 521. The program indicates the second calibration will start in 5 seconds 535 as part the system customization accommodation threshold 533. The user then looks at either their hand or a book at reading position 531. The program determines if second stage of calibration customization is good 537. If the second stage of calibration customization is bad, then the user must restart the calibration customization process 521. Once the program acknowledges that the second stage of calibration customization is good 537 the system has the completed customization accommodation calibration and the ophthalmic devices are ready for full use by the user.

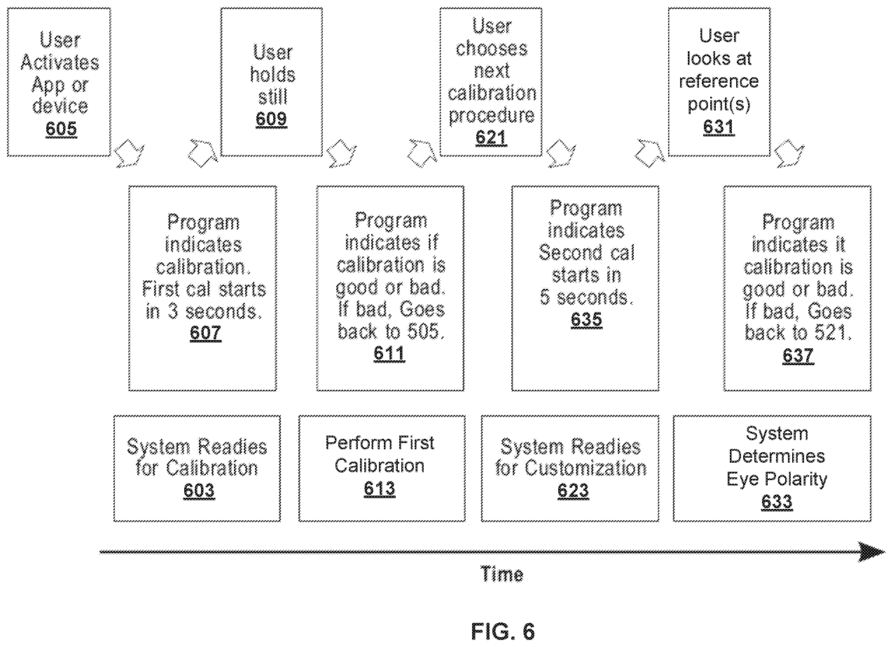

Referring to FIG. 6, another method according to an embodiment of the present invention is depicted. Once the ophthalmic devices (see FIG. 3) are inserted, the system readies for calibration 603. User activates App or device 605. The app program indicates calibration and the first calibration starts in 3 seconds 607 as part of a first calibration. The user holds still 609 as the system starts the first calibration 613. The first calibration can comprise pairing ophthalmic devices. The pairing can begin by determining, by a first ophthalmic device, presence of a second ophthalmic device. The first ophthalmic device can be caused to pair the first sensor system with the second sensor system in response to determining the presence of the second ophthalmic device. As part of paring, the first ophthalmic device can establish a secure communication channel based a security protocol. Pairing can further comprise synchronizing clocks (e.g., local oscillators) between the first sensor system (e.g., or processor thereof) and the second sensor system (e.g., or processor thereof).

The program indicates if first calibration is good or bad 611. If calibration is bad the program restarts and goes back (to step 605) 611. After the first calibration, the system is ready for a second calibration 623. The user may choose the next calibration procedure 621 or the second calibration procedure may be begin automatically. The program indicates the second calibration will start in 5 seconds 635 as part the system customization accommodation threshold 633. A calibration sequence can be provided to the user (e.g., via an app, via a mobile device). The calibration sequence can comprise one or more calibration instructions. Each calibration instruction can comprise one or more reference locations. The user then looks at one or more reference locations 631. The one or more reference locations can comprise a first reference location. The one or more reference locations can comprise a second reference location further away from the user than the first reference location. For example, the first reference location can comprise an object (e.g., book) close to the user (e.g., within a threshold distance, such as in the user's hand). The second reference location can comprise an object (e.g., wall) far from the user (e.g., located at least a threshold distance away from the user).

A polarity can be determined for each of the ophthalmic devices 533. The second stage of calibration can comprise a device polarity procedure for determining in which eye each of the ophthalmic devices are located. During the second stage of calibration one or more of the ophthalmic devices can receive sensor data. In some scenarios, step 631 can be performed at the time or can be the same step as step 531 of FIG. 5. For example, multiple calibrations can be performed based on the same instructions to a user to look at one or more reference locations.

One or more of a first sensor system of a first ophthalmic device and a second sensor system of a ophthalmic devices can receive sensor data (e.g., from one or more of the first sensor system or the second sensor system). The sensor data can be received during the calibration sequence for one or more of a first ophthalmic device or a second ophthalmic device (e.g., received during step 631). As explained further herein, the first sensor system can be disposed on or in the first ophthalmic device. The second sensor system can be disposed in or on the second ophthalmic device.

A change in a characteristic of one or more of the first eye or the second eye can be determined based on at least a portion of the sensor data. The change can relate to a calibration instruction provided to the user during the calibration sequence. For example, the change can represent a change from the user looking at the first reference point to looking at the second reference point. As a further explanation, a direction that the user moves one or more of the first eye or the second eye (e.g., during the calibration sequence) can be determined. A change in a vergence angle of one or more of the first eye or the second eye (e.g., during the calibration sequence) can be determined.

A polarity determination can be made for each of the ophthalmic devices. A first polarity indicating that the first ophthalmic device is disposed adjacent one of a right eye or a left eye can be determined based on the change in the characteristic of one or more of the first eye or the second eye. A second polarity indicating that the second ophthalmic device is disposed adjacent the other of the right eye or left eye can be determined. The second polarity can be based on at least a portion of the sensor data. The second polarity can also be determined based on the first polarity. A first identifier can be associated with the first ophthalmic device. A second identifier can be associated with the second ophthalmic device. The first identifier can be indicative of the first polarity and the second identifier can be indicative of the second polarity.

The program determines if the second stage of calibration customization is good 637. Such determination may be made by comparing resulting calibration offsets or calibration ratios to a range of reasonable values (determined from historic data or predetermined databases of offset information), or by comparing one or more resulting calibration offsets or calibration ratios to one or more other resulting calibration offsets or calibration ratios, for example, to determine if the difference between or ratio of the values falls within an acceptable or reasonable range (e.g., within a set tolerance of historical data or usage, or based on an error from a preset value). If so, the calibration can be complete. Additional sensor data can be processed based on one or more of the first polarity and the second polarity.

It should be noted that in another embodiment, the method can be performed by interacting with one or more of the ophthalmic devices directly, instead of via a program as shown in FIG. 6. For example, similar to the steps in FIG. 2, steps 607, 611, 635, and 637 can be performed by interaction with the first ophthalmic device and/or the second ophthalmic device. The ophthalmic device can acknowledge the first calibration and/or second calibration with activation of a component (e.g., lens) of the ophthalmic device. The user can perform a blink pattern to activate the first calibration and/or second calibration.

It is important to note that the above described elements may be realized in hardware, in software or in a combination of hardware and software. In addition, the communication channel may comprise any include various forms of wireless communications. The wireless communication channel may be configured for high frequency electromagnetic signals, low frequency electromagnetic signals, visible light signals, infrared light signals, and ultrasonic modulated signals. The wireless channel may further be used to supply power to the internal embedded power source acting as rechargeable power means.

The present invention may be a system, a method, and/or a computer program product. The computer program product being used by a controller for causing the controller to carry out aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method, comprising:
receiving, by one or more of a first sensor system and a second sensor system, sensor data during a calibration sequence for one or more of a first ophthalmic device or a second ophthalmic device, wherein the first sensor system is disposed on or in the first ophthalmic device and the second sensor system is disposed in or on the second ophthalmic device;
determining, based on the sensor data, a change in a characteristic of one or more of the first eye or the second eye, wherein the change relates to a calibration instruction provided to the user during the calibration sequence;
determining a first polarity indicating that the first ophthalmic device is disposed adjacent one of a right eye or a left eye based on the change in the characteristic of one or more of the first eye or the second eye;
determining a second polarity indicating that the second ophthalmic device is disposed adjacent the other of the right eye or left eye; and
associating a first identifier with the first ophthalmic device and a second identifier with the second ophthalmic device, wherein the first identifier is indicative of the first polarity and the second identifier is indicative of the second polarity.

2. The method of claim 1, wherein the calibration instruction comprises an instruction for the user to look at one or more reference locations.

3. The method of claim 2, wherein the one or more reference locations comprise a first reference location and a second reference location further away from the user than the first reference location.

4. The method of claim 1, wherein determining, based on the sensor data, the change in the characteristic of one or more of the first eye or the second eye comprises determining a direction that the user moves one or more of the first eye or the second eye.

5. The method of claim 1, wherein determining, based on the sensor data, the change in the characteristic of one or more of the first eye or the second eye comprises determining a change in a vergence angle of one or more of the first eye or the second eye.

6. The method of claim 1, further comprising providing the calibration instruction to the user via a mobile device.

7. The method of claim 1, further comprising:
determining, by the first ophthalmic device, presence of the second ophthalmic device; and
causing the first ophthalmic device to pair the first sensor system with the second sensor system in response to determining the presence of the second ophthalmic device.

8. The method of claim 1, further comprising processing additional sensor data based on one or more of the first polarity and the second polarity.

9. An ophthalmic system comprising:
a first ophthalmic device configured to be disposed adjacent a first eye of a user, the first ophthalmic device comprising a first sensor system, the first sensor system comprising a first sensor and a first processor operably connected to the first sensor; and
a second ophthalmic device configured to be disposed adjacent a second eye of the user, the second ophthalmic device comprising a second sensor system, the second sensor system comprising a second sensor and a second processor operably connected to the second sensor,
wherein one or more of the first processor or the second processor is configured to,
receive sensor data from one or more of the first sensor or the second sensor during a calibration sequence,
determine, based on the sensor data, a change in a characteristic of one or more of the first eye or the second eye, wherein the change relates to a calibration instruction provided to the user during the calibration sequence,
determine a first polarity indicating that the first ophthalmic device is disposed adjacent one of a right eye or a left eye based on the change in the characteristic of one or more of the first eye or the second eye,
determine a second polarity indicating that the second ophthalmic device is disposed adjacent the other of the right eye or left eye, and
associate a first identifier with the first ophthalmic device and a second identifier with the second ophthalmic device, wherein the first identifier is indicative of the first polarity and the second identifier is indicative of the second polarity.

10. The system of claim 9, wherein the calibration instruction comprises an instruction for the user to look at one or more reference locations.

11. The system of claim 10, wherein the one or more reference locations comprise a first reference location and a second reference location further away from the user than the first reference location.

12. The system of claim 9, wherein one or more of the first processor or the second processor being configured to determine, based on the sensor data, the change in the characteristic of one or more of the first eye or the second eye comprises one or more of the first processor or the second processor being configured to determine a direction that the user moves one or more of the first eye or the second eye.

13. The system of claim 9, wherein one or more of the first processor or the second processor is configured to determine, based on the sensor data, the change in the characteristic of one or more of the first eye or the second eye comprises one or more of the first processor or the second processor being configured to determine a change in a vergence angle of one or more of the first eye or the second eye.

14. The system of claim 9, wherein the calibration instruction is provided to the user via a mobile device.

15. The system of claim 9, wherein the first processor is configured to determine presence of the second ophthalmic device and cause the first ophthalmic device to pair the first sensor system with the second sensor system in response to determining the presence of the second ophthalmic device.

16. The system of claim 9, wherein one or more of the first processor or the second processor are configured to process additional sensor data based on one or more of the first polarity and the second polarity.

17. An ophthalmic system comprising:
a first ophthalmic device configured to be disposed adjacent at least one of a right eye of a user or a left eye of the user; and
a first sensor system disposed in or on the first ophthalmic device, the first sensor system comprising a first sensor and a first processor operably connected to the first sensor and configured to cause pairing of the first sensor system and a second sensor system disposed in or on a second ophthalmic device,
wherein the first processor is configured to determine whether the first ophthalmic device is disposed adjacent the left eye of the user or the right eye of the user based on a calibration sequence provided to the user.

18. The system of claim 17, wherein the calibration sequence comprises an instruction for the user to look at one or more reference locations.

19. The system of claim 18, wherein the one or more reference locations comprise a first reference location and a second reference location further away from the user than the first reference location.

20. The system of claim 17, wherein whether the first ophthalmic device is disposed adjacent the left eye of the user or the right eye of the user is determined based on a detecting a direction that the user moves one or more of the left eye or the right eye.

21. The system of claim 17, wherein whether the first ophthalmic device is disposed adjacent the left eye of the user or the right eye of the user is determined based on a detecting a change in a vergence angle of one or more of the left eye or the right eye.

22. The system of claim 17, wherein the calibration sequence is provided to the user via a mobile device.

23. The system of claim 17, wherein being configured to cause pairing of the first sensor system and the second sensor system comprises the first sensor system being configured to authenticate the second sensor system based on a security protocol.

24. The system of claim 17, wherein the first sensor system is configured to synchronize the first sensor system with the second sensor system.
